(19) United States
(12) Patent Application Publication
(10) Pub. No.: US 2026/0352991 A1
(43) Pub. Date: Nov. 5, 2026

(54) Flexible Sleeve Tip for an Earbud Having an Integrated Strain Gauge

(71) Applicant: Google LLC, Mountain View, CA (US)

(72) Inventors: Seamus David Thomson, Mountain View, CA (US); Peter W Richards, San Francisco, CA (US)

(21) Appl. No.: 19/487,231

(22) PCT Filed: May 24, 2023
(86) PCT No.: PCT/US2023/023314

§ 371 Date: Nov. 24, 2025

(51) Int. Cl. H04R 1/10; G01L 1/22; H04R 1/06

(52) U.S. Cl. CPC H04R 1/1016; G01L 1/22; H04R 1/06; H04R 1/1041; H04R 1/1058

(57) ABSTRACT

An earbud assembly includes an earbud housing containing one or more electrical components therein. The earbud housing defines a body. The earbud assembly also includes a flexible sleeve tip secured over at least a portion of the body. The flexible sleeve tip is configured to fit within an ear canal of a user. The earbud assembly further includes a strain gauge arranged with the flexible sleeve tip and being electrically connected to the one or more electrical components within the earbud housing via an electrical connection.

### FIELD

The present disclosure relates generally to audio devices compatible with wearable computing devices, and more particularly, to flexible sleeve tips for audio devices, such as earbuds, having an integrated strain gauge for jaw movement detection.

### BACKGROUND

In-ear headphones, or earbuds, are becoming increasingly popular due to their functionality, ease of use, and Bluetooth® capability with many computing devices. A design aspect of many earbuds is a flexible sleeve tip that fits over rigid components of the earbuds. As such, the flexible sleeve tip is designed to fit comfortably within an ear canal of a user. When the user opens and closes their mouth (such as when eating, yawning, smiling, etc.), the muscles and skeletal structures of the jaw move, which can cause the ear canal to change shape. As the ear canal adjusts in shape, the flexible sleeve tip is designed to conform so the earbuds remain in place and provide a good seal.

### SUMMARY

Aspects and advantages of embodiments of the present disclosure will be set forth in part in the following description, or can be learned from the description, or can be learned through practice of the embodiments.

In an aspect, the present disclosure is directed to an earbud assembly that includes an earbud housing containing one or more electrical components therein. The earbud housing defines a body, such as a rigid body. The earbud assembly also includes a flexible sleeve tip secured over at least a portion of the body. The flexible sleeve tip is configured to fit within an ear canal of a user. The earbud assembly further includes a strain gauge arranged with the flexible sleeve tip and being electrically connected to the one or more electrical components within the earbud housing via an electrical connection.

In another aspect, the present disclosure is directed to a method of manufacturing an earbud assembly. The method includes arranging one or more electrical components within an earbud housing, the earbud housing defining a body. The method also includes placing a flexible sleeve tip over at least a portion of the body, the flexible sleeve tip configured to fit within an ear canal of a user. Further, the method includes arranging a strain gauge with the flexible sleeve tip. Moreover, the method includes electrically connecting the strain gauge to the one or more electrical components within the earbud housing via an electrical connection.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1 illustrates a pictorial diagram of an embodiment of earbuds in accordance with aspects of the disclosure.

FIG. 2 illustrates a functional block diagram of a control system for earbuds in accordance with aspects of the disclosure.

FIG. 3 illustrates a perspective view of an embodiment of an earbud assembly in accordance with aspects of the disclosure.

FIG. 4 illustrates a front view of an embodiment of an earbud assembly in accordance with aspects of the disclosure.

FIG. 5 illustrates a top view of an embodiment of an earbud assembly in accordance with aspects of the disclosure.

FIG. 6 illustrates a top view of another embodiment of an earbud assembly in accordance with aspects of the disclosure.

FIGS. 7A-7C illustrate front views of various embodiments of an earbud assembly in accordance with aspects of the disclosure.

FIG. 8 illustrates a front view of an embodiment of an earbud assembly in accordance with aspects of the disclosure.

FIGS. 9A-9C illustrate top views of various embodiments of an earbud assembly in accordance with aspects of the disclosure.

FIG. 10 illustrates a front view of an embodiment of an earbud assembly in accordance with aspects of the disclosure, particularly illustrating a strain gauge design for the earbud assembly.

FIG. 11 illustrates a front view of another embodiment of an earbud assembly in accordance with aspects of the disclosure, particularly illustrating a strain gauge design for the audio device.

FIG. 12 illustrates a flow diagram of an embodiment of a method of manufacturing an earbud assembly in accordance with aspects of the disclosure.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

### Overview

As mentioned, a design aspect of many earbuds is a flexible sleeve tip that fits over a rigid component of the earbuds. As such, the flexible sleeve tip is designed to fit comfortably within an ear canal of a user. When the user opens and closes their mouth (such as when eating, yawning, smiling, etc.), the muscles and skeletal structures of the jaw move, which-due to the elastic properties of the skin and cartilage of the ear canal-can cause the ear canal to change shape. As the ear canal adjusts in shape, the flexible sleeve tip is designed to conform so the earbuds remain in place and provide a good seal.

Accordingly, the present disclosure is directed to an earbud having a flexible sleeve tip that includes at least one integrated strain gauge (e.g., at least one strain gauge embedded or otherwise attached to the flexible sleeve tip). A strain gauge is a sensor that measures the deformation of an object. It is made of a thin, electrically conductive material that is usually bonded to the surface of an object. When the object is deformed, the electrical resistance of the strain gauge changes. This change in resistance can be measured and used to calculate the amount of deformation. By incorporating a strain gauge into the flexible sleeve tip, deformation of the ear canal, and consequently jaw movement, can be measured. Thus, the strain gauge(s) is configured to facilitate measurement of the displacement of the flexible sleeve tip within the ear, e.g., as the flexible sleeve tip conforms to the ear canal contraction and/or expansion. In addition, the strain gauge(s) is configured to facilitate the electrical connection to the rigid component(s) of the earbuds that contain the accompanying electrical architecture. As such, data sourced from the strain gauge(s) can be used by any suitable computing device for analysis, such as in health and/or wellness products and/or features that relate to jaw movement.

With reference now to the Figures, example embodiments of the present disclosure will be discussed in further detail.

### Example Devices and Systems

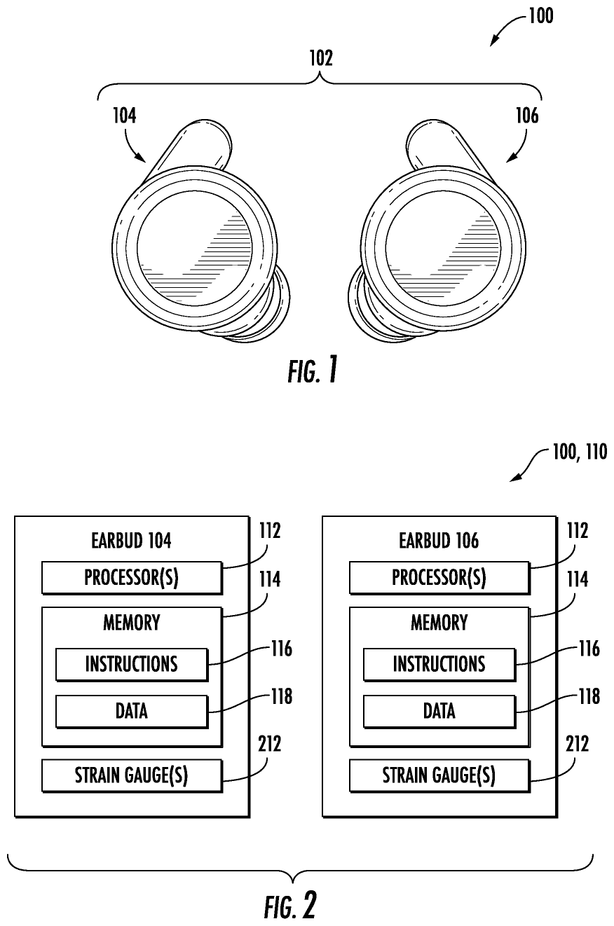

Referring now to the drawings, FIGS. 1-2 illustrate various views of a wearable computing device 100 according to the present disclosure. In an embodiment, the wearable computing device 100 may be a device that is capable of detecting and/or receiving audio input using one or more microphones. In an embodiment, the wearable computing device 100 may be earbuds, a smartwatch, a headset, smart glasses, a VR/AR headset, etc. In particular, as shown, the wearable computing device 100 is a pair 102 of earbuds 104, 106.

FIG. 2 illustrates a block diagram of an embodiment of a control system 110 in which the features described above and herein may be implemented. In the illustrated embodiment, for example, the control system 110 may include the earbuds 104, 106. Further, as shown, the control system 110 may contain one or more processors 112, memory 114, instructions 116, data 118, one or more strain gauges 120, and/or one or more microphones 122 associated with each of the earbuds 104, 106.

The processor(s) 112 may be any suitable processor, such as commercially available microprocessors. Alternatively or additionally, the processor(s) 112 may be a dedicated device such as an application specific integrated circuit (ASIC) or other hardware-based processor. Although FIG. 2 functionally illustrates the processor, memory, and other elements of the control system 110 as being within the same block, it will be understood by those of ordinary skill in the art that the processor, computing device, or memory may actually include multiple processors, computing devices, or memories that may or may not be stored within the same physical housing. Similarly, the memory 114 may be a hard drive or other storage media located in a housing different from that of the control system 110.

Accordingly, references to a processor or computing device will be understood to include references to a collection of processors or computing devices or memories that may or may not operate in parallel.

The memory 114 may store information that is accessible by the processor(s) 112, including the instructions 116 that may be executed by the processor(s) 112, and the data 118. The memory 114 may be a type of memory operative to store information accessible by the processor(s) 112, including a non-transitory computer-readable medium, or other medium that stores data that may be read with the aid of an electronic device, such as a hard-drive, memory card, read-only memory (“ROM”), random access memory (“RAM”), optical disks, as well as other write-capable and read-only memories. The present disclosure described herein may include different combinations of the foregoing, whereby different portions of the instructions 116 and the data 118 are stored on different types of media.

The memory 114 may be retrieved, stored, or modified by the processor(s) 112 in accordance with the instructions 116. For instance, although the present disclosure is not limited by a particular data structure, the data 118 may be stored in computer registers, in a relational database as a table having a plurality of different fields and records, XML documents, or flat files. The data 118 may also be formatted in a computer-readable format such as, but not limited to, binary values, ASCII or Unicode. By further way of example only, the data 118 may be stored as bitmaps comprising pixels that are stored in compressed or uncompressed, or various image formats (e.g., JPEG), vector-based formats (e.g., SVG) or computer instructions for drawing graphics. Moreover, the data 118 may include information sufficient to identify the relevant information, such as numbers, descriptive text, proprietary codes, pointers, references to data stored in other memories (including other network locations) or information that is used by a function to calculate the relevant data.

The instructions 116 can be any set of instructions to be executed directly, such as machine code, or indirectly, such as scripts, by the processor(s) 112. In that regard, the terms “instructions,” “application,” “steps,” and “programs” can be used interchangeably herein.

The instructions 116 can be stored in object code format for direct processing by the processor(s) 112, or in any other computing device language including scripts or collections of independent source code modules that are interpreted on demand or compiled in advance.

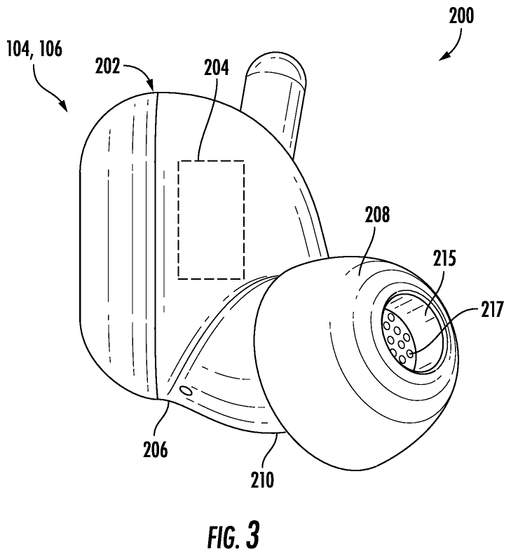

The wearable computing device 100 may further include one or more strain gauges 212 as will be further explained herein with reference to FIGS. 3-11. For example, as shown in FIG. 2, the control system 110 of the wearable computing device 100 may include one or more strain gauges 120 communicatively coupled to the processors(s) 112 described herein.

Referring now to FIGS. 3-11, various views of embodiments of an earbud assembly 200 containing one of the earbuds 104, 106 described herein according to the present disclosure are illustrated. In particular, FIG. 3 illustrates a perspective view of an embodiment of an earbud assembly 200 containing one of the earbuds 104, 106 described herein according to the present disclosure. As shown, the earbud assembly 200 includes an earbud housing 202 containing one or more electrical components 204 therein. For example, in an embodiment, the electrical component(s) 204 may include a driver, a speaker, a microphone, wiring, etc. Further, in an embodiment, the earbud housing 202 defines a body 206, such as a rigid body.

In addition, as shown in FIGS. 3 and 4, the earbud assembly 200 includes a flexible sleeve tip 208 secured over at least a portion of the body 206. In an embodiment, the flexible sleeve tip 208 may be constructed of flexible materials including but not limited to silicone, rubber, polymers such as memory foam (polyurethane), etc. As such, the flexible sleeve tip 208 is configured to fit within an ear canal of a user. In particular embodiments, for example, the body 206 may include at least one protrusion 210 extending therefrom. Thus, in such embodiments, as shown, the flexible sleeve tip 208 may be secured over at least a portion of the protrusion 210. More particularly, as shown generally in FIGS. 3-10, the protrusion 210 may define an opening 215 covered by an earbud cover 217.

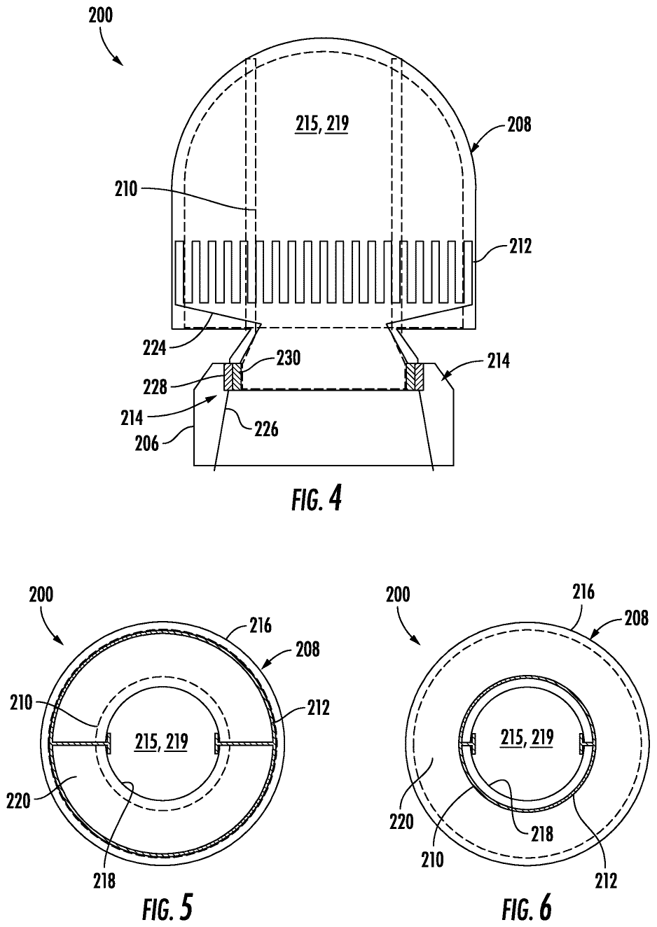

Referring now to FIGS. 3-11, the earbud assembly 200 further includes at least one strain gauge 212 arranged with the flexible sleeve tip 208. In such embodiments, the strain gauge(s) 212 can be constructed of any suitable conductive material (e.g., such as a conductive ink). For example, in an embodiment, the conductive materials may have similar mechanical properties to the flexible sleeve tip 208 (e.g., such as the conductive polymer poly(3,4-ethylenedioxythiophene)). Further, as shown in FIGS. 4, 7A, 7B, 7C, 8, 10, and 11, the strain gauge(s) 212 is electrically connected to the electrical component(s) 204 within the earbud housing 202 via an electrical connection 214. In an embodiment, as shown in FIG. 5, the strain gauge(s) 212 may be arranged on or adjacent to an exterior surface 216 of the flexible sleeve tip 208. In another embodiment, as shown in FIG. 6, the strain gauge(s) 212 may be arranged on or adjacent to an interior surface 218 of the flexible sleeve tip. In further embodiments, as shown in FIGS. 5 and 6, the strain gauge(s) 212 may be embedded at least partially within a wall of the flexible sleeve tip 208.

Furthermore, as shown generally in FIGS. 3-11, the strain gauge(s) 212 may be arranged concentrically around the flexible sleeve tip 208, i.e., following the cylindrical nature of the flexible sleeve tip 208. For example, as shown in FIGS. 4-6, the strain gauge(s) 212 may be arranged concentrically around the opening 215 defined by the protrusion 210.

Moreover, as shown in FIGS. 4-6, the flexible sleeve tip 208 also generally defines an opening 219 that aligns with the opening 215 of the protrusion. Thus, as shown, the strain gauge(s) 212 may be arranged concentrically around the opening 219 defined by the flexible sleeve tip 208.

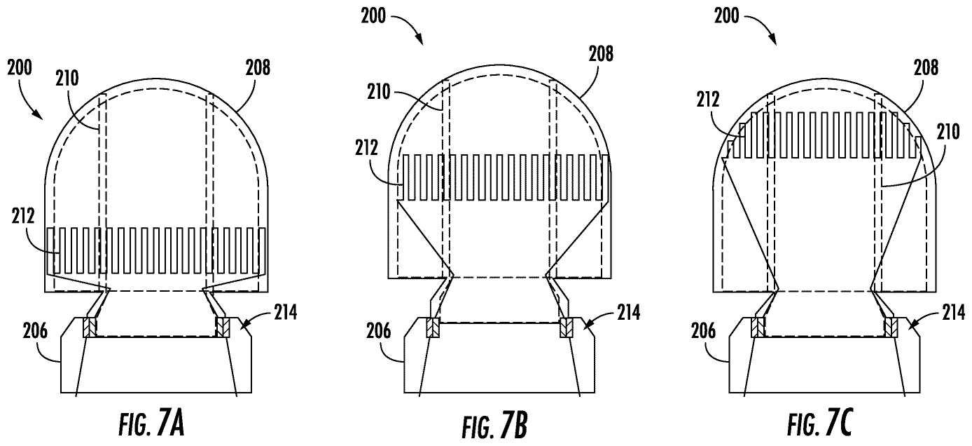

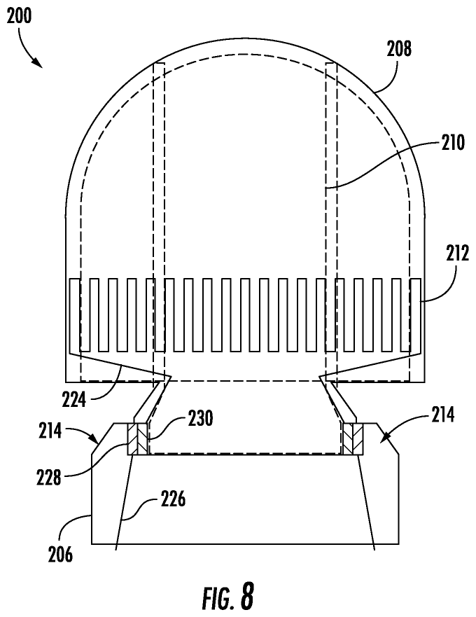

In further embodiments, the strain gauge(s) 212 may be arranged concentrically around at least one of a lower section of the flexible sleeve tip, an intermediate section of the flexible sleeve tip, an upper section of the flexible sleeve tip, or combinations thereof. In particular, as shown in FIG. 7A, the strain gauge(s) 212 may be arranged concentrically around a lower section of the flexible sleeve tip 208. In an embodiment, as shown in FIG. 7B, the strain gauge(s) 212 may be arranged concentrically around an intermediate section of the flexible sleeve tip 208. In an embodiment, as shown in FIG. 7C, the strain gauge(s) 212 may be arranged concentrically around an upper section of the flexible sleeve tip 208. In still another embodiment, any combination of FIGS. 7A-7C may also be utilized for arranging the strain gauge(s) 212 with respect to the flexible sleeve tip 208.

The strain gauge(s) 212 illustrated generally in the figures is not necessarily drawn to scale or at the correct density, or represented in the necessary direction where it may be optimal for measuring strain. Thus, the strain gauge(s) 212 described herein are provided for illustrative purposes only. Accordingly, in an embodiment, the location of the strain gauge(s) 212 may be more lateral (FIG. 5) or medial (FIG. 6) to the center of the flexible sleeve tip 208. The location of the strain gauge(s) 212 may also vary within the longitudinal direction (e.g., top and bottom) of the flexible sleeve tip 208 (FIGS. 7A-7C).

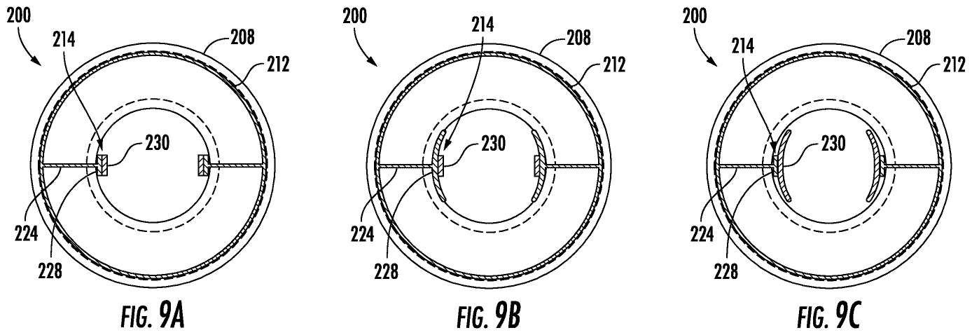

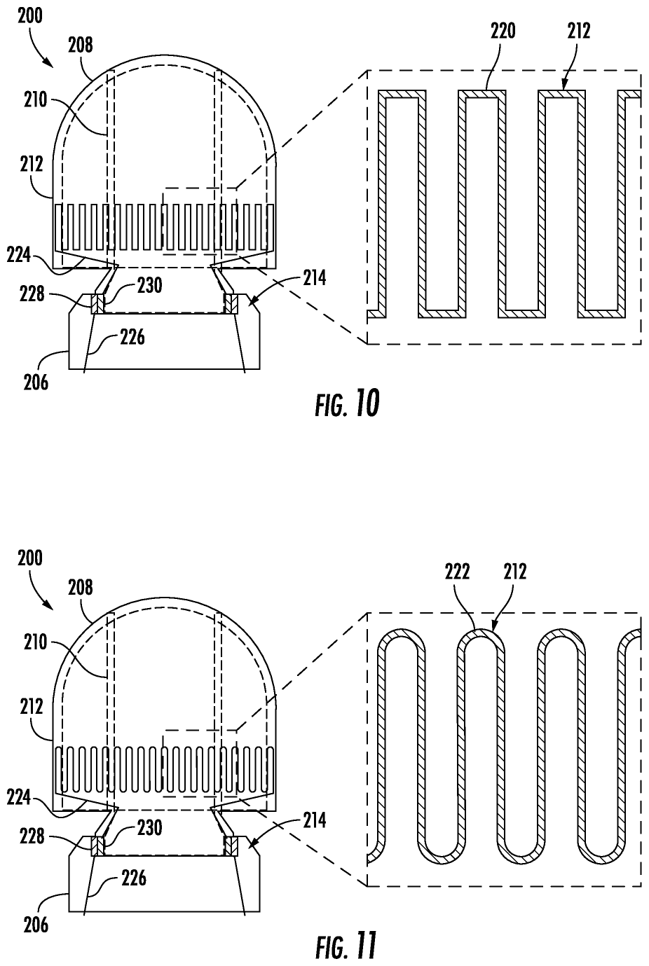

Several depictions on how the strain gauge(s) 212 electrically connects to the body 206 of the earbud assembly 200 are shown in FIGS. 8-9C. In particular, as shown, conductive material connects to the strain gauge(s) 212 that travels down the flexible sleeve tip 208 to the location where the flexible component mechanically connects to the body 206. At the location of this mechanical connection, there is an electrical connection 214 between the rigid and flexible components that may vary in the area/geometry of conductive materials to ensure a constant electrical connection (FIGS. 9A-9C). Strain gauge geometry may also vary/not be of homogenous pattern throughout the flexible sleeve tip 208, to help optimize how the strain gauge(s) 212 may impact stress distribution throughout the component (FIGS. 10 and 11).

Referring particularly to FIGS. 4, 8-11, the electrical connection 214 of the earbud assembly 200 may include first conductive wiring 224 electrically coupled to the strain gauge(s) 212 and second conductive wiring 226 electrically coupled to the electrical component(s) 204. Further, in an embodiment, as shown, the first and second conductive wirings 224, 226 are electrically coupled together at the electrical connection 214 via respective conductive ends 228, 230. Accordingly, in an embodiment, the respective conductive ends 228, 230 may include a first end 228 of the first conductive wiring 224 and a second end 230 of the second conductive wiring 226. Thus, in an embodiment, as shown in FIG. 9A, the first and second ends 228, 230 may each have the same geometry.

In another embodiment, as shown in FIGS. 9B and 9C, the first and second ends 228, 230 may each have different geometry. For example, as shown in FIG. 9B, the first end 228 of the first conductive wiring 224 electrically coupled to the strain gauge(s) 212 may be larger than the second end 230 of the second conductive wiring 226 electrically coupled to the electrical component(s) 204. Moreover, as shown, a shape of the first end 228 of the first conductive wiring 224 electrically coupled to the strain gauge(s) 212 may correspond to a shape of an interior surface of the flexible sleeve tip 208.

In another embodiment, as shown in FIG. 9C, the first end 228 of the first conductive wiring 224 electrically coupled to the strain gauge(s) 212 may be smaller than the second end 230 of the second conductive wiring 226 electrically coupled to the electrical component(s) 204. Moreover, as shown, a shape of the second end 230 of the second conductive wiring 226 electrically coupled to the electrical component(s) 204 may correspond to a shape of an interior surface of the flexible sleeve tip 208.

Referring now to FIGS. 10 and 11, the strain gauge(s) 212 may be arranged in an oscillating pattern. For example, as shown in FIG. 10, the oscillating pattern may be a step-up-step-down pattern 220. In another embodiment, as shown in FIG. 11, the oscillating pattern may be an undulating pattern 222.

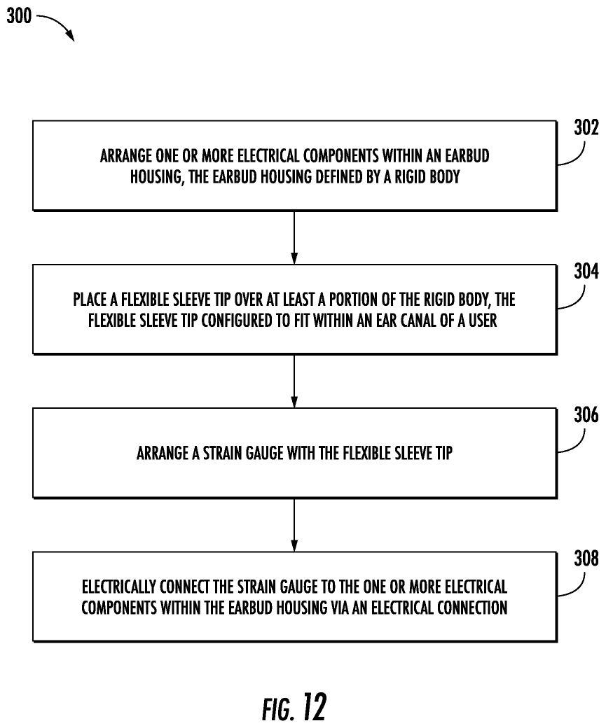

Referring now to FIG. 12, a flow diagram of one embodiment of a method 300 of manufacturing an earbud assembly, such as earbud assembly 200, is provided. In an embodiment, for example, the earbud assembly may be any suitable earbud assembly such as the earbud assembly 200 described herein with reference to FIGS. 1-11. In general, the method 300 is described herein with reference to the earbud assembly 200 of FIGS. 1-11. However, it should be appreciated that the disclosed method 300 may be implemented with any other suitable earbud assembly having any other suitable configurations. In addition, although FIG. 12 depicts steps performed in a particular order for purposes of illustration and discussion, the methods discussed herein are not limited to any particular order or arrangement. One skilled in the art, using the disclosures provided herein, will appreciate that various steps of the methods disclosed herein can be omitted, rearranged, combined, and/or adapted in various ways without deviating from the scope of the present disclosure.

As shown at (302), the method 300 includes arranging one or more electrical components within an earbud housing, the earbud housing defining a body, such as a rigid body. As shown at (304), the method 300 includes placing a flexible sleeve tip over at least a portion of the body. Further, as shown, the flexible sleeve tip is configured to fit within an ear canal of a user. Moreover, the flexible sleeve tip may include a strain gauge. For example, in an embodiment, as shown at (306), the method 300 may include arranging the strain gauge with the flexible sleeve tip. As shown at (308), the method 300 includes electrically connecting the strain gauge to the one or more electrical components within the earbud housing via an electrical connection.

In an embodiment, the method 300 may further include forming the body of the earbud housing with at least one protrusion extending therefrom. In such embodiments, the method 300 may include securing the flexible sleeve tip over at least a portion of the protrusion. Moreover, in an embodiment, arranging the strain gauge with the flexible sleeve tip may include arranging the strain gauge on an exterior surface of the flexible sleeve tip, an interior surface of the flexible sleeve tip, and/or embedded at least partially within a wall of the flexible sleeve tip. In addition, arranging the strain gauge with the flexible sleeve tip may include arranging the strain gauge concentrically around the flexible sleeve tip.

### Additional Disclosure

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

What is claimed is:

1. An earbud assembly, comprising:
an earbud housing containing one or more electrical components therein, the earbud housing defining a body;
a flexible sleeve tip secured over at least a portion of the body, the flexible sleeve tip configured to fit within an ear canal of a user; and
a strain gauge arranged with the flexible sleeve tip and electrically connected to the one or more electrical components within the earbud housing via an electrical connection.

2. The earbud assembly of claim 1, wherein the body comprises at least one protrusion extending therefrom, the flexible sleeve tip secured over at least a portion of the at least one protrusion.

3. The earbud assembly of claim 1, wherein the strain gauge is arranged on at least one of an exterior surface of the flexible sleeve tip, an interior surface of the flexible sleeve tip, or embedded at least partially within a wall of the flexible sleeve tip.

4. The earbud assembly of claim 1, wherein the strain gauge is arranged concentrically around the flexible sleeve tip.

5. The earbud assembly of claim 4, wherein the strain gauge is arranged concentrically around at least one of a lower section of the flexible sleeve tip, an intermediate section of the flexible sleeve tip, an upper section of the flexible sleeve tip, or combinations thereof.

6. The earbud assembly of claim 1, wherein the strain gauge is arranged in an oscillating pattern.

7. The earbud assembly of claim 6, wherein the oscillating pattern is at least one of a step-up-step-down pattern or an undulating pattern.

8. The earbud assembly of claim 1, wherein the electrical connection comprises first conductive wiring electrically coupled to the strain gauge and second conductive wiring electrically coupled to the one or more electrical components, the first and second conductive wirings electrically coupled together at the electrical connection via respective conductive ends.

9. The earbud assembly of claim 8, wherein the respective conductive ends comprise a first end of the first conductive wiring and a second end of the second conductive wiring, the first and second ends each comprising the same geometry.

10. The earbud assembly of claim 8, wherein the respective conductive ends comprise a first end of the first conductive wiring and a second end of the second conductive wiring, the first and second ends each comprising different geometry.

11. The earbud assembly of claim 10, wherein the first end of the first conductive wiring electrically coupled to the strain gauge is smaller than the second end of the second conductive wiring electrically coupled to the one or more electrical components.

12. The earbud assembly of claim 11, wherein the second end of the second conductive wiring electrically coupled to the one or more electrical components corresponds to a shape of an interior surface of the flexible sleeve tip.

13. The earbud assembly of claim 10, wherein the first end of the first conductive wiring electrically coupled to the strain gauge is larger than the second end of the second conductive wiring electrically coupled to the one or more electrical components.

14. The earbud assembly of claim 13, wherein the first end of the first conductive wiring electrically coupled to the strain gauge corresponds to a shape of an interior surface of the flexible sleeve tip.

15. A method of manufacturing an earbud assembly, the method comprising:
arranging one or more electrical components within an earbud housing, the earbud housing defining a body;
placing a flexible sleeve tip over at least a portion of the body, the flexible sleeve tip having a strain gauge, the flexible sleeve tip configured to fit within an ear canal of a user; and
electrically connecting the strain gauge to the one or more electrical components within the earbud housing via an electrical connection.

16. The method of claim 15, further comprising forming the body of the earbud housing with at least one protrusion extending therefrom, the flexible sleeve tip secured over at least a portion of the at least one protrusion.

17. The method of claim 15, further comprising arranging the strain gauge with the flexible sleeve tip, wherein arranging the strain gauge with the flexible sleeve tip further comprises arranging the strain gauge on at least one of an exterior surface of the flexible sleeve tip, an interior surface of the flexible sleeve tip, or embedded at least partially within a wall of the flexible sleeve tip.

18. The method of claim 17, wherein arranging the strain gauge with the flexible sleeve tip further comprises arranging the strain gauge concentrically around the flexible sleeve tip.

19. The method of claim 18, wherein the strain gauge is arranged concentrically around at least one of a lower section of the flexible sleeve tip, an intermediate section of the flexible sleeve tip, an upper section of the flexible sleeve tip, or combinations thereof.

20. The method of claim 17, wherein arranging the strain gauge with the flexible sleeve tip further comprises arranging the strain gauge in an oscillating pattern.
